(19) United States
(12) Patent Application Publication
(10) Pub. No.: US 2004/0122416 A1
(43) Pub. Date: Jun. 24, 2004

(54) Locking guidewire straightener

(75) Inventors: Timothy Schweikert, Levittown, PA (US); Nicholas V. Gately, Lambertville, NJ (US)

Correspondence Address: MONTE & MCGRAW, PC, 4092 SKIPPACK PIKE, P.O. BOX 650, SKIPPACK, PA, 19474, US

(73) Assignee: Medical Components, Inc., Harleysville, PA

(21) Appl. No.: 10/366,247

(22) Filed: Feb. 13, 2003

Related U.S. Application Data
(60) Provisional application No. 60/434,262, filed on Dec. 18, 2002.

(51) Int. Cl. A61M 25/02

(52) U.S. Cl. 604/528; 600/434

(57) ABSTRACT

A catheter guidewire straightener is disclosed. The guidewire straightener includes an elongated body having a distal end, a proximal end having an exterior surface with a diameter sized to fit within a catheter guidewire tube, and a longitudinal passageway sized to accept a catheter guidewire. The passageway extends between the proximal end and the distal end. The proximal end further includes at least one lug extending from the exterior surface and a biasing member operatively connected to each of the at least one lug and biasing the at least one lug away from the passageway such that, when the proximal end is disposed within the guidewire tube, the biasing member biases the at least one lug against the guidewire tube.

### CROSS-REFERENCE TO RELATED APPLICATION

The present application claims priority from U.S. Provisional Patent Application Serial No. 60/434,262, filed on Dec. 18, 2002.

### FIELD OF THE INVENTION

The present invention relates to a catheter guidewire and a method of retaining a catheter guidewire in a guidewire tube.

### BACKGROUND OF THE INVENTION

Catheter guidewires are used to facilitate introduction of a catheter into a patient. In a typical catheter installation, an insertion site is located, and an incision is made by the inserting physician at the insertion site. A syringe is inserted into the patient through the incision to locate the blood vessel, such as a vein, to be catheterized. A flexible metal guidewire is typically inserted through the syringe and into the blood vessel to further enable the inserting physician to insert the catheter into the patient. The guidewire typically has a “J-shaped” distal end, so as not to tear into the patient's blood vessel during insertion. The guidewire is quite flexible so that it can bend with the contours of the blood vessel, but the flexibility of the guidewire, along with the “J-shape”, can make it difficult for the physician to insert the distal end of the guidewire into the syringe. Guidewire straighteners are used to straighten out the “J-shape” and to assist the physician in inserting the distal tip of the guidewire into the syringe for advancement into the blood vessel.

A guidewire is typically stored in a coiled plastic tube, both for ease of physician use and for sterility. However, during shipping, the guidewire can work its way out of the tube, making the guidewire more difficult to handle and potentially compromising the sterility of the guidewire.

A known guidewire straightener is disclosed in U.S. Pat. No. 5,125,905. This guidewire straightener utilizes a cantilevered extension at the proximal end of the straightener to grasp a guidewire and retain the guidewire in its tube. It is believed by the inventor that the prior art design does not provide sufficient gripping between the straightener and the guidewire. It is also believed by the inventor that the prior art design may be prone to breakage from fatigue.

It would be beneficial to provide a guidewire straightener that provides increased gripping between the straightener and the guidewire so that the guidewire does not work its way out of the tube, as well as a guidewire straightener that is less prone to breakage from fatigue.

### BRIEF SUMMARY OF THE INVENTION

Briefly, the present invention provides a guidewire straightener. The guidewire straightener comprises an elongated body having a distal end, a proximal end having an exterior surface with a diameter sized to fit within a catheter guidewire tube, and a longitudinal passageway sized to accept a catheter guidewire. The passageway extends between the proximal end and the distal end. The proximal end further includes at least one lug extending from the exterior surface and a biasing member operatively connected to each of the at least one lug and biasing the at least one lug away from the passageway such that, when the proximal end is disposed within the guidewire tube, the biasing member biases the at least one lug against the guidewire tube.

Additionally, the present invention provides a catheter guidewire assembly. The guidewire assembly comprises a catheter guidewire tube having a distal end and a tube passageway extending therethrough and a guidewire straightener having a distal end, a proximal end disposed within the distal end of the guidewire tube and having at least one biasing assembly extending therefrom, and a straightener passageway extending between the proximal and distal ends of the straightener. The straightener passageway is co-axial with and communicates with the tube passageway. The guidewire assembly further comprises a catheter guidewire slidingly disposed within the guidewire tube passageway and the straightener passageway. The guidewire tube biases the at least one biasing assembly into the straightener passageway, such that the at least one biasing assembly frictionally engages the guidewire.

Further, the present invention provides a catheter guidewire assembly. The guidewire assembly comprises a catheter guidewire tube having a distal end and a tube passageway extending therethrough and a guidewire straightener having a distal end, a proximal end disposed within the distal end of the guidewire tube, a straightener passageway extending between the proximal and distal ends of the straightener, wherein the straightener passageway is co-axial with and communicates with the tube passageway, and gripping means on the proximal end for frictionally engaging the guidewire straightener within the guidewire tube. The guidewire assembly further comprises a catheter guidewire slidingly disposed within the guidewire tube passageway and the straightener passageway. The guidewire tube biases the gripping means into the straightener passageway, such that the gripping means longitudinally engages the guidewire.

Also, the present invention provides a method of retaining a catheter guidewire in a guidewire tube comprising providing a guidewire tube having a distal end; disposing a guidewire within the guidewire tube wherein a distal end of the guidewire extends from the distal end of the guidewire tube; and releasably inserting a proximal end of a guidewire straightener into the distal end of the guidewire tube, wherein the proximal end of the guidewire straightener includes at least one means for engaging the guidewire straightener with the guidewire tube, and wherein the means releasably engages the guidewire along a longitudinal length.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a perspective view of a catheter guidewire straightener according to an embodiment of the present invention.

FIG. 2 is a side elevational view of the catheter guidewire straightener shown in FIG. 1.

FIG. 3 is a sectional view of the catheter guidewire straightener taken along line 3-3 of FIG. 2.

FIG. 4 is a plan view of a catheter guidewire assembly incorporating the catheter guidewire straightener shown in FIGS. 1-3.

FIG. 5 is a sectional view of the catheter guidewire assembly taken along line 5-5 of FIG. 4.

FIG. 6 is a schematic view showing operation of the catheter guidewire assembly during insertion of a guidewire into a patient.

### DETAILED DESCRIPTION OF THE INVENTION

In the drawings, like numerals indicate like elements throughout. Certain terminology is used herein for convenience only and is not to be taken as a limitation on the present invention. The words “proximal” and “distal” refer to directions away from and closer to, respectively, the insertion tip of the guidewire in the guidewire assembly according to the present invention. The terminology includes the words above specifically mentioned, derivatives thereof, and words of similar import. The following describes a preferred embodiment of the invention. However, it should be understood based on this disclosure, that the invention is not limited by the preferred embodiment described herein.

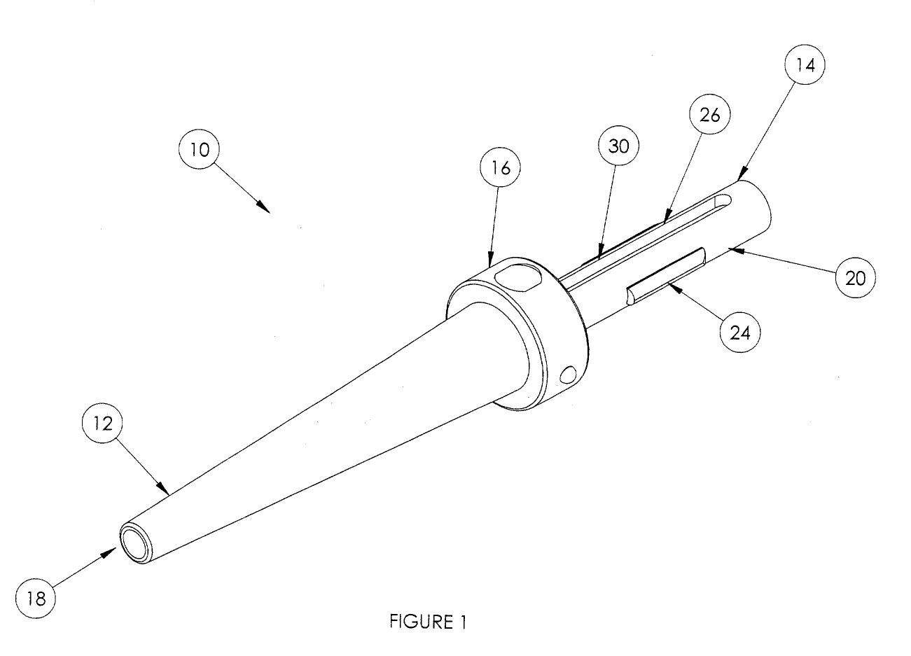

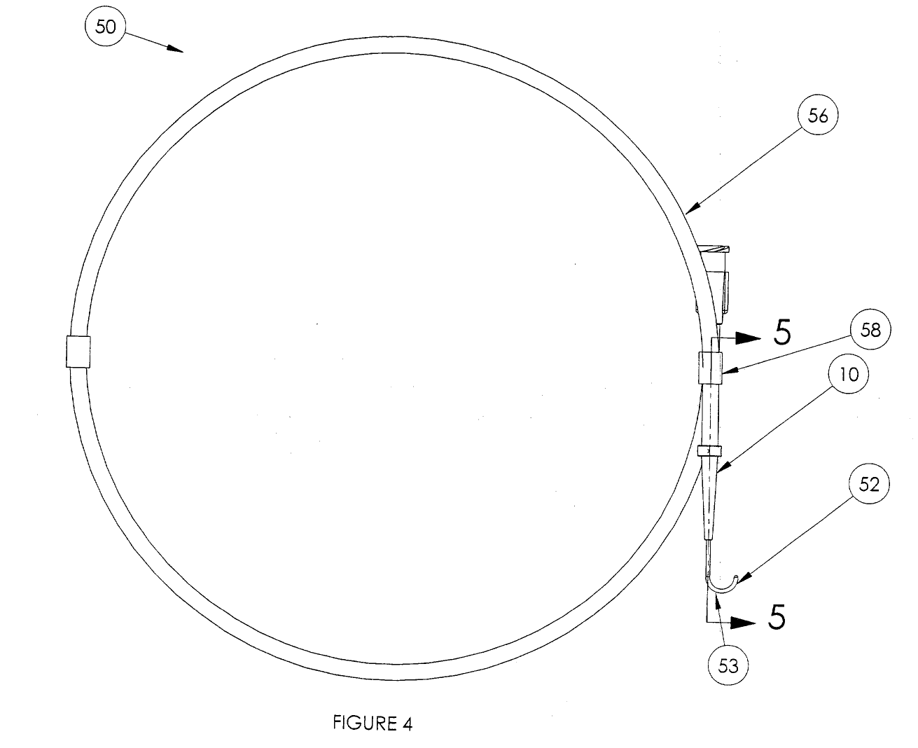

A catheter guidewire straightener 10 according to an embodiment of the present invention is shown in FIGS. 1-3. The guidewire straightener 10 is comprised of a generally frusto-conically shaped distal end 12 and a generally cylindrically shaped proximal end 14. A stop collar 16 is disposed between the distal end 12 and the proximal end 14. A straightener passageway 18 exntends through the guidewire straightener 10 between the distal end 12 and the proximal end 14. The straightener passageway 18 is sized to allow a guidewire 52, shown in a guidewire assembly 50 in FIG. 4, to pass through the straightener passageway 18 with little or no interference. A standard guidewire has a diameter of approximately 0.038 inches (approximately 0.97 mm), and the straightener passageway 18 has a diameter of approximately 0.05 inches (approximately 1.27 mm). Therefore, when the guidewire 52 is inserted into the straightener passageway 18, a clearance of approximately 0.012 inches (approximately 0.30 mm) is provided between the guidewire 52 and the guidewire straightener 10.

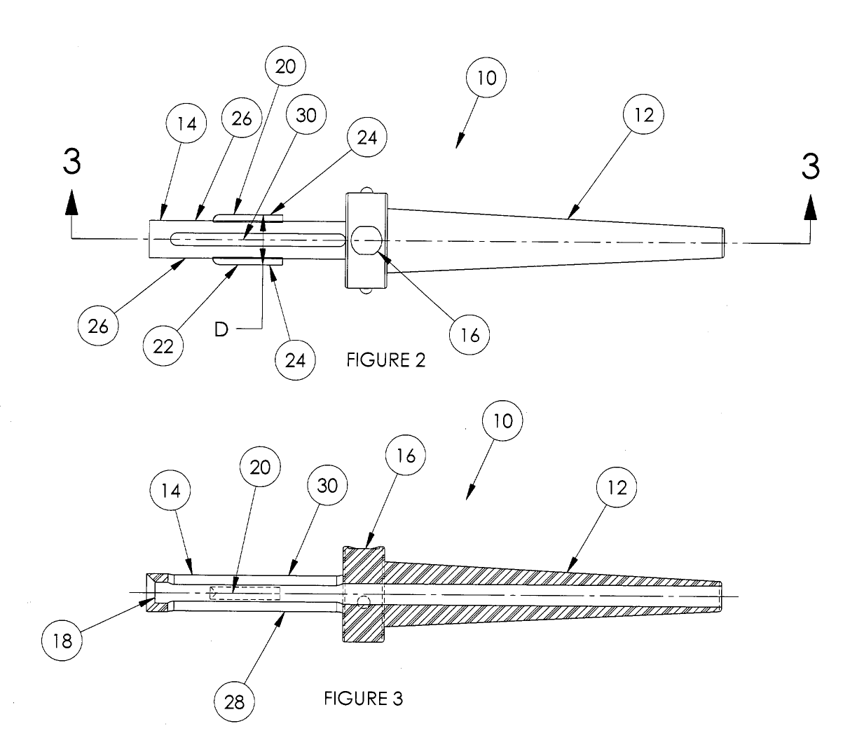

Referring back to FIGS. 1-3, the proximal end 14 of the straightener 10 includes first and second diametrically opposed biasing assemblies 20, 22, which are each comprised of a lug 24 disposed on a biasing member 26, such as a leaf spring. A distance between free ends of each lug 24 on the biasing assemblies 20, 22, as shown in FIG. 2, is defined as a diameter “D”.

Each biasing assembly 20, 22, extends longitudinally along the proximal end 14 of the straightener 10, with channels 28, 30 disposed on either side of the biasing assemblies 20, 22. The channels 28, 30 allow the lugs 24 and the biasing members 26 to deflect toward the straightener passageway 18 when force is applied on the lugs 24 toward the straightener passageway 18. The biasing members 26 resist such force and bias the lugs 24 away from the straightener passageway 18.

Preferably, the guidewire straightener 10 is constructed from a polymer, such as polypropylene, although those skilled in the art will recognize that other, suitable materials may be used.

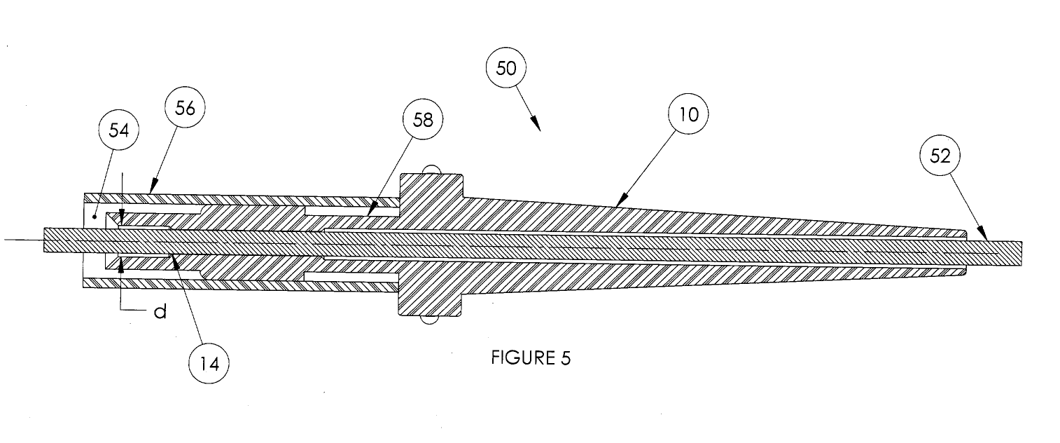

A guidewire assembly 50 incorporating the guidewire straightener 10 of the present invention is shown in FIGS. 4 and 5. The guidewire assembly 50 is comprised of a guidewire 52 disposed within a tube passageway 54 of a guidewire tube 56. The guidewire 52 has a “J-shaped” distal end 53. The tube passageway 54 has an inner diameter “d”. The guidewire tube 56 is connected to the proximal end 14 of the guidewire straightener 10.

The guidewire tube 56 is preferably coiled as shown for the ease of the user. The guidewire 52 is typically approximately 24 inches (61 cm) long and, by coiling the guidewire 52 along with the guidewire tube 56, the user can more readily handle the guidewire 52 and advance the guidewire 52 into the patient.

The guidewire tube 56 includes an open distal end 58, through which the guidewire 52 is advanced. The distal end 58 of the guidewire tube 56 is inserted over the proximal end 14 of the guidewire straightener 10, so that the distal end 58 of the guidewire tube 56 and the proximal end of the guidewire straightener 10 engage each other with a press fit.

The diameter “D” of the lugs 24 is larger than the diameter “d” of the tube passageway 54 so that, as is seen in FIG. 5, the guidewire tube 56 biases the lugs 24 and the biasing members 26 into the straightener passageway 18. Each lug 24 biases its respective biasing member 26 against the guidewire 52 so that the guidewire 52 is longitudinally engaged along a length of the guidewire 52 by the biasing members 26. The biasing members 26, in turn, bias the lugs 24 against the tube 56 to releasably retain the straightener 10 in the tube 56.

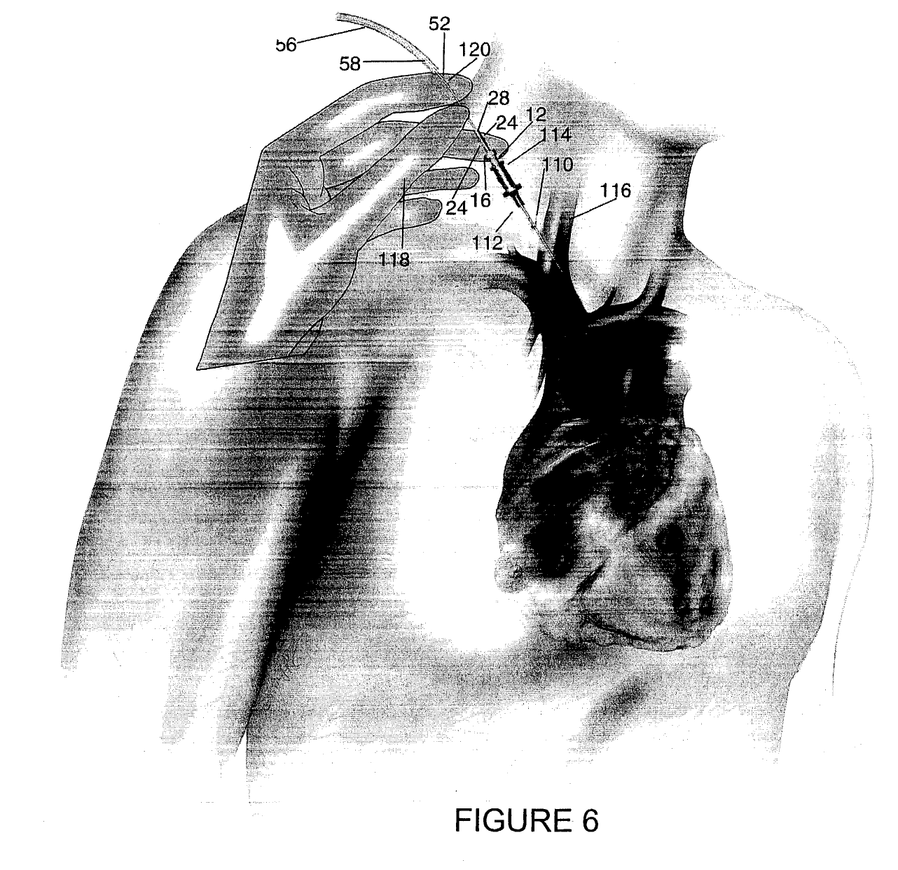

Referring now to the guidewire insertion procedure of FIG. 6 during a catheter insertion procedure, an incision 110 is initially made near an insertion site 112 which is to be aspirated with a syringe or other introducer apparatus near or proximate the area to be catheterized. If the catheter is used for hemodialysis and the area to be catheterized is the internal jugular vein 116, the incision 110 is made in the clavicular triangle region, as shown for example, in FIG. 6. The exact location of the incision 110 can be varied by the physician. In accordance with the Seldinger technique, a narrow needle 114 connected to the syringe is inserted through the incision 110 and into the vein 116, and the vein 116 is aspirated. The syringe is disconnected from the needle 114, leaving the needle 114 in the vein 116.

The guidewire 52 is next inserted into the vein 116 through the needle 114. To insert the guidewire 52 into the vein 116, the proximal end 14 of the straightener 10 is removed from the tube 56. The biasing members 26 bias away from the straightener passageway 18, releasing the guidewire 52 from between the biasing members 26. The distal end 53 of the guidewire 52 is then retracted into the straightener 10 so that the “J-shaped” distal end 53 of the guidewire 52 is disposed within the straightener 10. The distal end 12 of the straightener 10 is then inserted into the proximal end of the needle 114. The preferably frusto-conical shape of the distal end 12 facilitates insertion of the distal end 12 into the needle 114.

Using thumb 118 and forefinger 120 on the guidewire 52 between the straightener 10 and the tube 56, the physician advances the guidewire 52 through the needle 114 and into the vein 116. Once the guidewire 52 is in place, the tube 56 and the straightener 10 are removed by advancing each of the tube 56 and the straightener 10 along the guidewire 52 away from the distal end 53 of the guidewire 52 and then off the proximal end of the guidewire 52. Next, the needle 114 is removed by advancing the needle 114 along the guidewire 52 away from the distal end 53 of the guidewire 52 and then off the proximal end of the guidewire 52. A dilator (not shown) and a tearable sheath (not shown) are introduced over the guidewire 52 and partially into the vein 116. The insertion site 112 is now ready to accept a catheter assembly (not shown). At least one catheter lumen is disposed over the proximal end of the guidewire 52 and advanced toward the distal end 53 of the guidewire 52 and into the vein.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. A guidewire straightener comprising:
an elongated body having:
a distal end;
a proximal end having an exterior surface with a diameter sized to fit within a catheter guidewire tube; and
a longitudinal passageway sized to accept a catheter guidewire, the passageway extending between the proximal end and the distal end;
wherein the proximal end further includes at least one lug extending from the exterior surface and a biasing member operatively connected to each of the at least one lug and biasing the at least one lug away from the passageway such that, when the proximal end is disposed within the guidewire tube, the biasing member biases the at least one lug against the guidewire tube.

2. The guidewire straightener according to claim 1, wherein the distal end is generally frusto-conically shaped.

3. The guidewire straightener according to claim 1, wherein, when the guidewire is inserted into the guidewire tube, the biasing member frictionally engages a guidewire disposed in the passageway.

4. The guidewire straightener according to claim 1, further comprising a collar disposed on the body between the distal end and the proximal end.

5. The guidewire straightener according to claim 1, wherein the distal end is sized to fit within a syringe needle.

6. The guidewire straightener according to claim 1, wherein the at least one lug comprises a plurality of lugs.

7. The guidewire straightener according to claim 1, wherein the at least one lug comprises a first lug and a second lug disposed diametrically across the passageway from the first lug.

8. A catheter guidewire assembly comprising:
a catheter guidewire tube having a distal end and a tube passageway extending therethrough;
a guidewire straightener having:
a distal end;
a proximal end disposed within the distal end of the guidewire tube and having at least one biasing assembly extending therefrom; and
a straightener passageway extending between the proximal and distal ends of the straightener, wherein the straightener passageway is co-axial with and communicates with the tube passageway; and
a catheter guidewire slidingly disposed within the guidewire tube passageway and the straightener passageway;
wherein the guidewire tube biases the at least one biasing assembly into the straightener passageway, such that the at least one biasing assembly frictionally engages the guidewire.

9. The catheter guidewire assembly according to claim 8, wherein the distal end of the guidewire straightener is generally frusto-conically shaped.

10. The catheter guidewire assembly according to claim 8, wherein the guidewire straightener further comprises a collar disposed between the proximal end of the guidewire straightener and the distal end of the guidewire straightener.

11. The catheter guidewire assembly according to claim 8, wherein the at least one biasing assembly comprises a plurality of biasing assemblies.

12. The catheter guidewire assembly according to claim 8, wherein the at least one biasing assembly comprises a first biasing assembly and a second biasing assembly disposed diametrically across the straightener passageway from the first biasing assembly.

13. The catheter guidewire assembly according to claim 8, wherein the biasing assembly comprises a lug disposed on a biasing member.

14. The catheter guidewire assembly according to claim 13, wherein the biasing member is a leaf spring.

15. A catheter guidewire assembly comprising:
a catheter guidewire tube having a distal end and a tube passageway extending therethrough;
a guidewire straightener having:
a distal end;
a proximal end disposed within the distal end of the guidewire tube;
a straightener passageway extending between the proximal and distal ends of the straightener, wherein the straightener passageway is co-axial with and communicates with the tube passageway; and
gripping means on the proximal end of the guidewire straightener for frictionally engaging the guidewire straightener within the guidewire tube;
and
a catheter guidewire slidingly disposed within the guidewire tube passageway and the straightener passageway;
wherein the guidewire tube biases the gripping means into the straightener passageway, such that the gripping means longitudinally engages the guidewire.

16. The catheter guidewire assembly according to claim 15, wherein the gripping means comprises a lug disposed on a biasing member, wherein the lug engages the guidewire tube and the biasing member engages the guidewire.

17. A method of retaining a catheter guidewire in a guidewire tube comprising:
providing a guidewire tube having a distal end;
disposing a guidewire within the guidewire tube wherein a distal end of the guidewire extends from the distal end of the guidewire tube; and
releasably inserting a proximal end of a guidewire straightener into the distal end of the guidewire tube, wherein the proximal end of the guidewire straightener includes at least one means for engaging the guidewire straightener with the guidewire tube, and wherein the means releasably engages the guidewire along a longitudinal length.
